(19) United States
(12) Patent Application Publication
(10) Pub. No.: US 2008/0033251 A1
(43) Pub. Date: Feb. 7, 2008

(54) SURGICAL RETRACTOR AND METHOD OF USE

(76) Inventor: Ali Araghi, Scottsdale, AZ (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/772,668

(22) Filed: Jul. 2, 2007

Related U.S. Application Data
(60) Provisional application No. 60/818,024, filed on Jun. 30, 2006.

(51) Int. Cl. A61B 1/32

(52) U.S. Cl. 600/235; 600/201; 600/245

(57) ABSTRACT

A surgical retractor may be provided for a surgical procedure such as spinal surgery. The surgical retractor may include a pair of tissue retainers and a pair of separators. The separators may be positioned in channels of the tissue retainers to move distal ends of the tissue retainers apart, retract tissue, and enlarge an opening in a patient. In some embodiments, a nerve root retractor may be removably coupled to a surgical retractor to retain dura on one side of the spinal column and provide working room for the surgical procedure.

### PRIORITY CLAIM

This application claims priority to U.S. Provisional Patent Application 60/818,024 entitled “SURGICAL RETRACTOR” to Ali Araghi filed Jun. 30, 2006, which is incorporated by reference in its entirety.

### BACKGROUND

1. Field of the Invention

The present invention relates generally to surgical retractors. More particularly, the invention relates to a surgical retractor that may be used during a minimally invasive procedure. The surgical retractor may include tissue retainers that allow for a larger opening at a distal end of the retractor than at a proximal end of the retractor when the tissue retainers are separated.

2. Description of Related Art

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae (typically twenty-four) separated by intervertebral discs. The intervertebral discs hold the vertebrae together in a flexible manner to allow relative movement between the vertebrae from front to back and from side to side. This movement allows the body to bend forward and backward, to bend from side to side, and to rotate about a vertical axis. When the spine is operating properly, the nerves are maintained clear of the hard structure of the spine throughout the available ranges of motion.

Over time or because of accidents or disease, the intervertebral discs may lose height or become cracked, dehydrated, or herniated. The result is that the height of one or more discs may be reduced. The reduction in height can lead to compression of the nerve bundles. Such compression may cause pain and, in some cases, damage to the nerves.

Currently, there are many systems and methods at the disposal of a physician for reducing or eliminating the pain by minimizing the stress on the nerve bundles. In some instances, the existing disc is removed and an artificial disc is substituted therefore. In other instances, two or more vertebrae are fused together to prevent relative movement between the fused discs.

Often there is required a system and method for maintaining or recreating proper space for the nerve bundles that emerge from the spine at a certain location. In some cases, a cage or bone graft is placed in the disc space to preserve or restore height and to aid in fusion of the vertebral level. As an aid in stabilizing the vertebrae, one or more rods or braces are placed between the fused vertebrae with the purpose of supporting the vertebrae while the vertebrae fuse. The rods or braces are usually placed along the posterior of the spine. These rods and braces may be held in place by anchors that are placed in the pedicles of the vertebrae.

Traditional surgical procedures to correct injuries, defects, and/or abnormalities of the spine have been substantially invasive. To access the affected area of the spine, substantial incisions, extensive muscle stripping, prolonged retraction of tissues, denervation and/or devascularization of tissue have generally been required. Access to the affected area may cause significant trauma to the affected tissue and nearby nerves. Traditional open surgical procedures pose significant risks because the need to access locations deep within the body risks damage to vital intervening tissues including nerves, arteries, veins, muscles and/or ligaments. For example, open spinal surgeries have involved complications including but not limited to injury to the nerve root and dural sac, perineural scar formation, and reherniation at the surgical site. Recovery from the trauma to the tissue and nerves may cause significant pain to the patient and may require a long recovery period.

Minimally invasive surgical procedures have been developed to fuse or otherwise treat vertebrae. Minimally invasive surgical procedures are less invasive and require smaller incisions. Such procedures can reduce pain, post-operative recovery time, and the destruction of healthy tissue. Generally, a surgical site is accessed through portals, rather than through a significant incision, to aid in preserving the integrity of the intervening tissues. Minimally invasive surgical procedures are particularly desirable for spinal and neurosurgical applications because of the need for access to locations deep within the body and the possible range of damage to vital intervening tissues. In such procedures, however, it may be necessary to hold the edges of an incision apart to provide a clear operating field within which the surgeon can operate and to allow for the insertion of instruments and implants.

What is needed is a device capable of being inserted into a small incision which will retain tissue away from the incision opening to create a working space that provides a surgeon with a good view of the surgical site and a clear path to the operating field for the insertion of instruments and implants.

### SUMMARY

Some embodiments described herein are related to a surgical retractor. The surgical retractor includes a first tissue retainer and second tissue retainer. Each tissue retainer includes angled channels. The surgical retractor also includes at least one separator. A first portion of a separator is configured to couple to an angled channel of a first tissue retainer. A second portion of the separator is configured to couple to an angled channel of the second tissue retainer. The separator is configured to move a distal end of the first tissue retainer away from a distal end of the second tissue retainer when the separator is moved down the angled channels of the first tissue retainer and the second tissue retainer.

In some embodiments, the surgical retractor includes a nerve root retractor configured to couple to a separator. In some embodiments, the surgical retractor includes a nerve root retractor that is configured to couple to the first tissue retainer. In some embodiments, the surgical retractor includes an optical cable configured to couple an illumination source to the surgical retractor.

In some embodiments, a retractor system for a spinal surgery procedure is described. The retractor system may include a surgical retractor configured to enlarge a surgical opening in a patient, and a nerve root retractor configured to removably couple to the surgical retractor to allow the retention of dura of a patient to one side of a spinal column. The surgical retractor may include a pair of tissue retainers and a pair of separators.

Some embodiments described herein relate to a method of retraction during surgery. The method includes placing a pair of tissue retainers in an opening in a patient, coupling at least one separator to the tissue retainers, and moving the separator downwards to separate distal ends of the tissue retainers, retract tissue and form a larger opening.

### BRIEF DESCRIPTION OF THE DRAWINGS

Features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts a perspective view of a portion of an embodiment of a surgical retractor.

FIG. 2 depicts a perspective view of an embodiment of tissue retainers of a surgical retractor.

FIG. 3 depicts a cross-sectional representation of tissue retainers taken substantially along plane B-B of FIG. 2.

FIG. 4 depicts a top view of an embodiment of tissue retainers.

FIG. 5 depicts a perspective view of an embodiment of tissue retainers coupled together by an elastic member.

FIG. 6 depicts a front view of an embodiment of tissue retainers.

FIGS. 7-10 depict perspective views of embodiments of channels for tissue retainers.

FIG. 11 depicts a side view of an embodiment of a tissue retainer with an extender in an initial position.

FIG. 12 depicts a side view of the embodiment of the tissue retainer depicted in FIG. 11 with the extender moved downwards to lengthen the tissue retainer.

FIG. 13 depicts a perspective view of an embodiment of an extender separated from the tissue retainer.

FIG. 14 depicts a front view of an embodiment of a separator of a surgical retractor.

FIG. 15 depicts a side view of an embodiment of a surgical retractor.

FIG. 16 depicts a front view of an embodiment of a separator of a surgical retractor.

FIG. 17 depicts a side view of an embodiment of a nerve root retractor that may be used with the separator depicted in FIG. 16.

FIG. 18 depicts a side view of an embodiment of a nerve root retractor that may be used with the separator .depicted in FIG. 16.

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF EMBODIMENTS

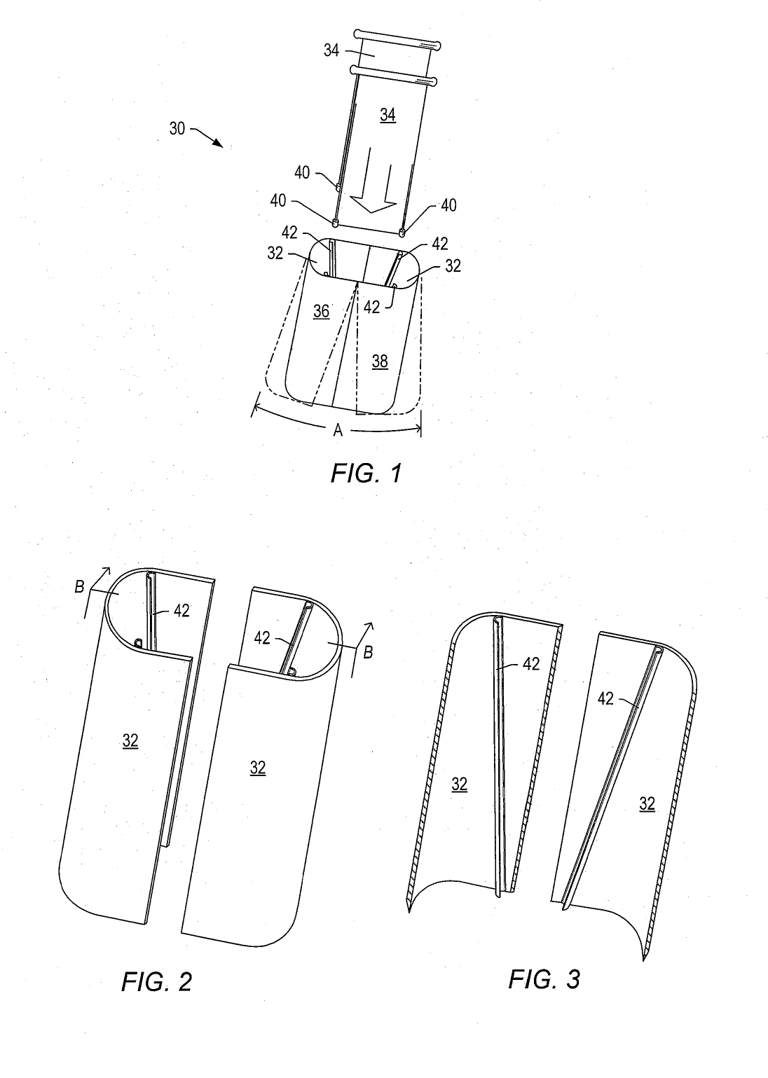

FIG. 1 depicts an embodiment of a surgical retractor. Surgical retractor 30 may include tissue retainers 32 and separators 34. All of surgical retractor 30, or portions of the surgical retractor 30 that are positioned in the patient, may be made of radiolucent material so that imaging techniques may be used while the surgical retractor is positioned in the patient. Surgical retractor 30 may be used during a surgical procedure to retract tissue of a patient to provide a surgeon access to an operating field. In some embodiments, surgical retractor 30 is used during spinal surgery. Surgical retractor 30 may also be used during other types of surgical procedures. Surgical retractor 30 may allow for a relatively small opening to be formed in the patient to accommodate needed access to the surgical site during the surgical procedure. Tissue retainers 32 may be inserted in a surgical incision in the patient. Separators 34 may be coupled to tissue retainers 32 and driven downwards to rotate the tissue retainers, enlarge the incision in the patient, and retract tissue. When separators 34 are driven downwards, the opening at the distal end of surgical retractor 30 may be larger than the opening at the proximal end of the surgical retractor. Providing a larger opening at the distal end of the surgical retractor may advantageously provide a large working area and proved good visualization of the operating field.

Surgical retractor 30 may be inserted in an incision in a patient in an initial position. In the initial position, edges of first tissue retainer 36 and second tissue retainer 38 are close together or touching, as represented by the solid lines of FIG. 1. Separators 34 may be inserted into surgical retractor 30. Guides 40 of separators 34 may be positioned in channels 42 of tissue retainers 36, 38. Separators 34 may be moved down channels 42 to spread first tissue retainer 36 away from second tissue retainer 38 and create a large working area. Separators 34 may spread distal ends of tissue retainers 36, 38 apart while the proximal ends of the tissue retainers remain close together, as represented by the dashed lines of FIG. 1. In some embodiments, separators 34 rotate tissue retainers 36, 38 and create an Angle A between the tissue retainers. In some embodiments, insertion of the separators between the tissue retainers moves the tissue retainers apart with little or no rotation resulting in little or no rotation of the tissue retainers. In other embodiments, insertion of the separators between the tissue retainers causes the tissue retainers to move about a moving center of rotation. The incision in the patient may remain relatively small as compared with traditional open spinal surgeries, and thereby cause less trauma to surrounding tissue and muscles.

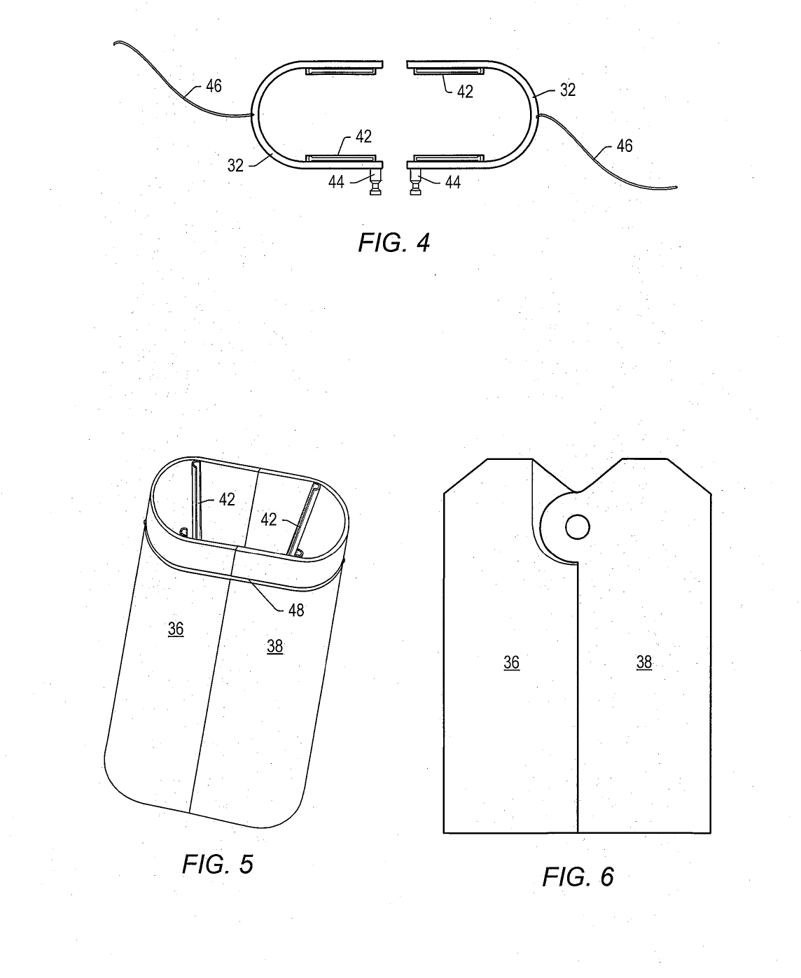

FIG. 2 depicts a perspective view of an embodiment of tissue retainers 32. FIG. 3 depicts a cross-sectional view of tissue retainers 32, taken substantially along plane B-B of FIG. 2. FIG. 4 depicts a top view of an embodiment of tissue retainers 32. Tissue retainers 32 may include channels 42. Channels 42 may be angled down the inner surface of tissue retainers 32. Angled channels 42 allow distal ends of tissue retainers 32 to move apart when the separators are positioned in the channels and moved downwards.

In some embodiments, angled channels 42 extend along the length of the tissue retainers. In other embodiments, angled channels 42 extend along only a portion of the length of tissue retainers 32. When the separators are fully inserted in channels 42, the upper surfaces of the guides of the separators may engage the bottom of the channels to inhibit undesired backout of the separators from the tissue retainers. When the surgical procedure is completed, a tool may be inserted down one or more of the channels to disengage the end of the channels from the tops of the guides to allow for removal of the separators from the tissue retainers. In other embodiments, other systems may be used to inhibit backout of the separator from the tissue retainers. For example a spring detent in the separator (or channel) may be positioned in a recess in the channel (or separator) when the separator is fully inserted in the tissue retainers. The spring detent may inhibit undesired backout of the separator from the tissue retainers. The separator may be forcefully moved upwards to release the spring detent from the recess and allow for removal of the separator from the tissue retainers. In other embodiments, a fastener (e.g., a latch or screw) may be used to couple the separator to the tissue retainers after the separator is fully inserted between the tissue retainers.

As depicted in FIG. 4, tissue retainers 32 may include pins 44. Pins 44 may be located near the proximal ends of tissue retainers 32. In some embodiments, the pins may be located on both sides of the tissue retainers. Pins 44 may couple to arms of a table mount. Arms and table mounts may be available from Mediflex Surgical Products (Islandia, N.Y.). The arms are adjustable to fix the positions of tissue retainers 32 relative to the patient. Once the position of tissue retainers 32 are properly positioned, the table mount and/or arms may be set to fix the position of the tissue retainers. When the arms of the table mount are set, tissue retainers 32 may still rotate to accommodate insertion of the separators in the channels 42 of the tissue retainers. In some embodiments, the arms of the table mount substantially fix the vertical position of the tissue retainers relative to the patient and allow horizontal movement of the tissue retainers relative to the patient to accommodate insertion of the separators.

Light sources may be coupled to the retractor to provide illumination to the operating field with little or no shadows. In some embodiments, a tissue retainer includes retaining channels. A light mat may slide down the retaining channel. Light may be supplied from a light source to the light mat through an optical cable. The light mat may be used to illuminate the operating area. In some embodiments, light rings may be coupled to the distal ends of the tissue retainers to provide illumination to the operating field. In other embodiments, the light source may be coupled or integrated with the separators.

In some embodiments, one or more of the tissue retainers and/or the separators are made of translucent material. For example, the tissue retainers and/or the separators may be made of polycarbonate or other high strength, translucent polymers. In some embodiments, the tissue retainers and/or the separators may include channels that accept and retain light mats that are positioned in the channels and lighted by light transmitted through optical cables. In some embodiments, optical cables may be coupled to tissue retainers and/or separators. In certain embodiments, the outer surface of the tissue retainer may include a coating or material that reflects or inhibits diffusion of light. The material that forms the inner surface of the tissue retainers may include material that diffuses light so that light supplied to the tissue retainers illuminates the operating field. Light from a light source provided through the fiber optic cable may illuminate the tissue retainers and provide illumination to the operating area. FIG. 4 depicts an embodiment of tissue retainers 32 with optical cables 46 coupled to the tissue retainers.

In some embodiments, tissue retainers may be coupled together using a member, members or wrapping. The member, members, or wrapping may be placed in grooves formed in the tissue retainers, may be held by fasteners or stops, and/or be otherwise coupled to the tissue retainers. FIG. 5 depicts tissue retainers 36, 38 that are coupled together by elastic member 48. Elastic member 48 may be replaced after use of the surgical retractor. In some embodiments, two or more members may be used to couple the tissue retainers together. In some embodiments, the tissue retainers may be initially coupled together by a member, members or wrapping during insertion of the tissue retainers in the patient. The member, members or wrapping may be removed before the separators are used to move the tissue retainers apart.

In some embodiments, tissue retainers may be coupled together near proximal ends of the tissue retainers. The tissue retainers may be hinged or otherwise coupled together. FIG. 6 depicts a front view of an embodiment of tissue retainers 36, 38 that are coupled together. A pin may couple first tissue retainer 36 to second tissue retainer 38. In some embodiments, one or more pins of the surgical retractor may extend out from tissue retainers 36, 38. A pin that extends out from the tissue retainers may be coupled to an arm of a table mount that fixes the position of the surgical retractor relative to the patient. Inner portions of the proximal ends of tissue retainers 36, 38 may be chamfered or angled to avoid restrictive contact of tissue retainer 36 with tissue retainer 38. Outer portions of the proximal ends of tissue retainers 36, 38 maybe angled or chamfered so that the proximal opening of the surgical retractor remains large when the separator rotates the tissue retainers.

In other embodiments, the tissue retractors may be coupled in other ways. For example, each tissue retainer may include a pin that extends into an elongated curved opening formed in the other tissue retainer. When the separators are moved down the grooves in the tissue retainers, the distal ends of the tissue retainers move away from each other. The tissue retainers remain coupled together and the paths of the tissue retainers are defined by the paths of the pins in the elongated curved openings.

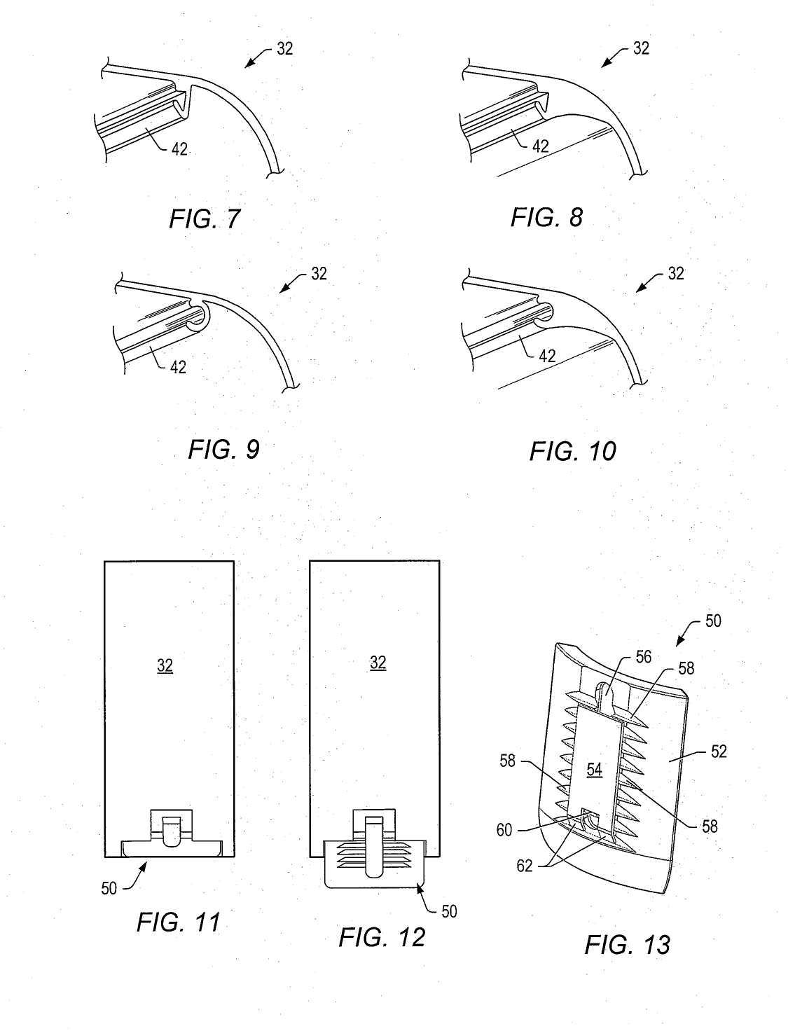

FIGS. 7-10 depict embodiments of shapes of channels that may be formed in tissue retainers. In addition to the illustrated shapes, other shapes may also be used and are within the scope of certain embodiments. As shown in FIG. 7 and FIG. 8, channel 42 may have a dovetail shape. As shown in FIG. 9 and FIG. 10, channels 42 may have an arced shape. In some embodiments, the portion of tissue retainer 32 that defines the back of channel 42 extends from the inner surface of the tissue retainer, as depicted in FIG. 7 and FIG. 9. In some embodiments, the portion of tissue retainer 32 that defines the back of channel 42 is formed as part of the inner surface of the tissue retainer, as depicted in FIG. 8 and FIG. 10. The guides of the separators may have shapes that complement the shapes of the channels.

As depicted in FIGS. 7-10, channels may include recessed portions that guides of the separators are positioned into allow the tissue retainers to be moved apart by the separators. In some embodiments, the channels include protrusions that fit within recesses in the guides of the separators to allow the tissue retainers to be moved apart by the separators.

When the separators are inserted in the tissue retainers, rotation of the tissue retainers may cause the distal ends of the tissue retainers to rise relative to the patient. In some embodiments, the tissue retainers or separators may include one or more extenders that allow the length of the tissue retainers or separators to be increased during the surgical procedure. FIG. 11 depicts a side view of an embodiment of tissue retainer 32 with extender 50 in an initial position. In the initial position, a distal end of extender 50 is substantially even with the distal end of the body of tissue retainer 32. FIG. 12 depicts an embodiment of tissue retainer 32 with extender 50 moved downwards to provide extra length to tissue retainer 32. Extender 50 may be reset to the initial position after the surgical retractor has been removed from the patient.

FIG. 13 depicts a perspective view of an embodiment of extender 50 separated from the tissue retainer. As illustrated, extender 50 may include extender body 52 and ratchet 54. In certain embodiments, sides of extender body 52 may fit in a groove in the body of the tissue retainer. Ratchet 54 may be secured to the inner surface of the tissue retainer.

Extender body 52 may include guide 56 and grooves 58. Grooves 58 may be cut in the body to have a sloping upper surface and a substantially vertical bottom surface. Ratchet 54 may include post 60 and arms 62. Post 60 may be positioned in guide 56. Post 60 and guide 56 may limit the travel distance of extender body 52 relative to the tissue retainer. Ratchet arms 62 may be positioned in a groove of grooves 58. An end of an adjustor may contact the uppermost surface of extender body to move extender body downwards relative to the tissue retainer. The sloping upper surfaces of grooves 58 allow the extension body to move downwards and extend from the body of the tissue retainer. When extender body 52 is moved downwards relative to the body of the tissue retainer, ratchet arms 62 are positioned in a different groove. Retraction of extender body 52 into the body of the tissue retainer is inhibited by contact of ratchet arms 62 with a substantially vertical bottom surface of groove 58.

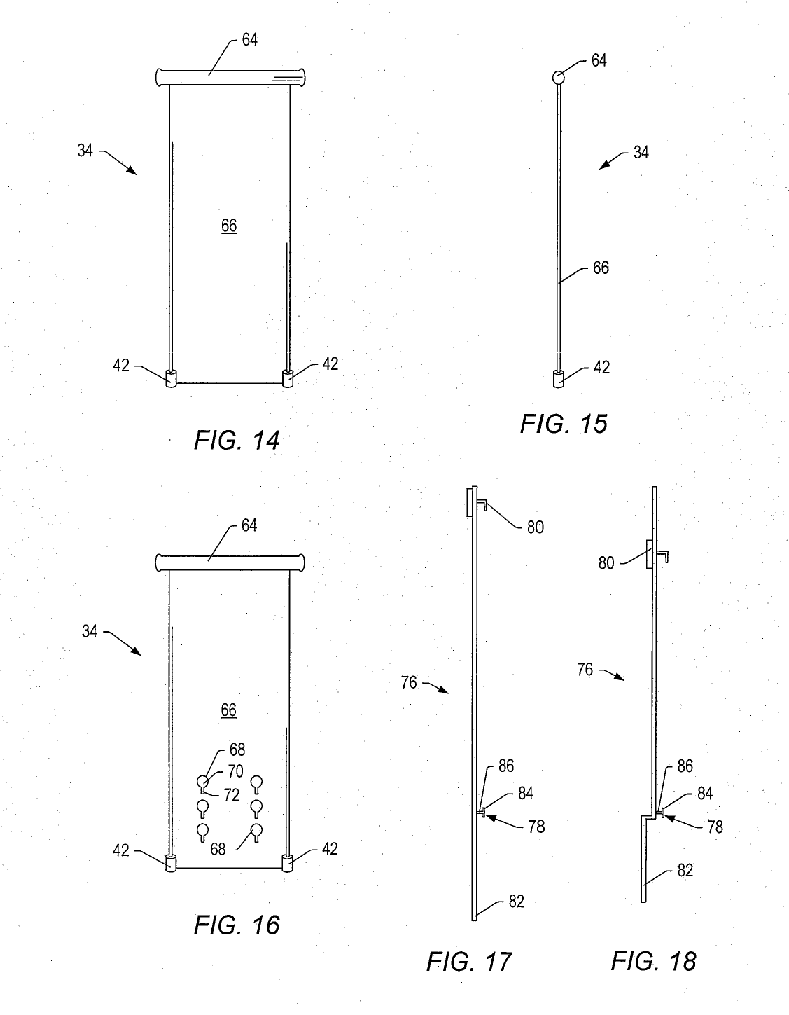

FIG. 14 depicts a front view of an embodiment of separator 34. FIG. 15 depicts a side view of the embodiment of separator 34. In the illustrated embodiment, separator 34 includes guides 40, bar 64, and body 66. Guides 40 may be positioned in the channels of the tissue retainers. Bar 64 may provide a grip for separator 34 and a surface for driving the separator down the channels of the tissue retainers. Body 66 may serve as a tissue retaining barrier when separator 34 is inserted into the tissue retainers.

In other embodiments, the separator may have a profile that is non-rectangular. For example, the separator may have a triangular, rhombic, trapezoidal, or irregularly shaped profile. In embodiments where the separators have trapezoidal shapes, distal ends of the separators may be larger than proximal ends. Such a shape may allow for the formation of a large angle (i.e., angle A in FIG. 1) between the tissue retainers of the surgical retractor.

Two separators 34 may be positioned in the channels of the tissue retainers. In some embodiments, a bridge may be coupled to bars 64 of separators 34 to join the separators together. When separators 34 are positioned in the channels of the tissue retainers, force may be applied to the bridge to drive the separators downwards in the tissue retainers. The bridge may ensure that separators 34 are simultaneously widening both sides of the surgical retractor. After separators 34 are inserted in the tissue retainers, the bridge may be removed from the separators.

As previously discussed, in some embodiments, a light source may be coupled to separator 34 by an optical cable. The light source may illuminate a light mat or the light source may illuminate the separator. Light supplied to separator 34 by the light source may illuminate the operating field. The optical cable may be coupled to separator 34 after the separator has been fully inserted into the tissue retainers.

In some embodiments, a nerve root retractor may be coupled to the surgical retractor. The nerve root retractor allows the dura or nerves to be held on a first side of the spinal column so that the surgeon has greater access to the operating area. During the surgical procedure, the dura may be held on a first side of the spinal column during one or more periods of the surgical procedure, and the dura may be held on the other side of the spinal column during other periods of the surgical procedure.

In some embodiments, nerve root retractors may be removably coupled to the tissue retainers. In some embodiments, nerve root retractors may be removably coupled to the separators. The nerve root retractors allow for retraction of the dura without the need for a person to hold the dura in the retracted position. In other embodiments, the nerve root retractors may be thinner versions of the extenders discussed previously in reference to FIGS. 11 through 13.

FIG. 16 depicts an embodiment of separator 34 that allows a nerve root retractor to be coupled to the separator. Separator 34 may include one or more keyways 68. In some embodiments, the keyways may be formed in the tissue retainers of the surgical retractor. Keyway 68 may include opening 70 and channel 72. A nerve root retractor may include a protrusion with a head that fits in opening 70 and a shaft that fits in channel 72. The head may be placed in opening 70 and the nerve root retractor may be moved downwards so that the shaft is positioned in channel 72. When the shaft is positioned in channel 72, removal of the nerve root retractor from separator 34 is inhibited until the head aligns with opening 70. In an embodiment, the separator may only include one set of keyways. Similarly, other embodiments of the tissue retainers may have keyways configured in a similar fashion.

FIG. 17 and FIG. 18 depict certain embodiments of nerve root retractor 76 that may be used with .the separator depicted in FIG. 16 and/or tissue retainers. In some embodiments, nerve root retractor 76 may include protrusion 78, slidable hook 80, and holder 82. Protrusion 78 may include head 84 and shaft 86. Head 84 may be sized to fit in the keyway opening of the separator, and shaft 86 may fit in the keyway channel. Slidable hook 80 may contact the upper surface of the separator. The position of slidable hook 80 is adjustable so that single nerve root retractor 76 is able to accommodate being placed in any of the available keyways formed in the separator. Sliding hook 80 may be moved to an outermost position before protrusion 78 is placed in a keyway. After protrusion 78 is placed in the keyway, and nerve root retractor is moved downwards to place shaft 86 in the keyway channel, slidable hook 80 may be moved downwards to rest on the top of the separator.

Holder 82 may retain the dura in an out of the way location to provide access to a desired location during the surgical procedure. In certain embodiments, the holder may be about 3 mm to 10 mm wide with rounded edges. In some embodiments, such as the embodiment depicted in FIG. 17, nerve root retractor 76 is a relatively straight member, and the dura may be held below the distal end of the separator. In some embodiments, such as the embodiment depicted in FIG. 18, holder 82 is offset from an upper portion of nerve root retractor 76. In some embodiments, the holder may rotate or slide relative to the upper portion of the nerve root retractor. The holder may lock in one or more positions.

The surgical retractor may be provided in a kit. The kit may include a case that holds accessories, instruments, and the components of the surgical retractor. The case may have a plurality of openings. The entire case may be placed in a sterilizer to sterilize all of the contents within the case. Some of the contents in the case may be pre-sterilized and placed in bags that are put into the case. Accessories included in the case may include, but are not limited to, arms and table mounts, a table adaptor, light cables and adaptors, disposable light mats, and trays.

Instruments included in the case may include a dilator set, retraction devices, an adjustor and a driver. The dilator set may be used to expand the initial incision made in the patient. The driver may be used to rotate fasteners that couple the surgical retractor to the arms of the table mount. The retraction devices may include nerve root retractors.

The surgical retractor components may include the tissue retainers, separators and one or more nerve root retractors. The kit may include tissue retainers and separators that form surgical retractors of various lengths. For example tissue retainers and separators that form surgical retractors having lengths of 40 mm, 50 mm, 60 mm, 70 mm, 80 mm, 90 mm, 100 mm, and 110 mm may be included in the kit. Different component lengths and/or a different number of components may be supplied in the kit.

When using various embodiments of the surgical retractor, an incision may be formed in the patient. The incision may be expanded using the dilators. The tissue retainers may be positioned in the incision on the outside of the largest dilator. The tissue retainers may be coupled to arms of a table mount .system. The table mount system may be used to fix the position of the tissue retainers. The dilator may be removed from the patient. Separators may be positioned in the channels of the tissue retainers. The separators may be moved downwards to expand the incision. In some embodiments, an adjustor may be used to move extenders of the tissue retainers downwards.

In some embodiments, light mats may be coupled to the surgical retractor to provide illumination for the surgical procedure. Optical cables may be coupled to the light mats to provide light to the light mats. In some embodiments, optical cables may be coupled directly to one or more components of the surgical retractor to provide illumination for the surgical procedure.

During some spinal procedures, the dura may be retracted to one side of the spinal column using a retraction device. The slidable hook may be moved to an outermost position The protrusion of the nerve retractor may be inserted in an appropriate keyway of the surgical retractor. The nerve root retractor may be moved downwards to inhibit undesired separation of the nerve root retractor from the surgical retractor. The slidable hook may be moved downwards to rest on top of the surgical retractor. The retraction device may be removed.

After the surgical procedure is completed, optical cables may be disconnected. The separators may be removed from the tissue retainers. The tissue retainers may be released from the table mount, and the tissue retainers may be removed from the patient.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A surgical retractor, comprising:
a first tissue retainer, the first tissue retainer having one or more angled channels;
a second tissue retainer, the second tissue retainer having one or more angled channels; and
one or more separators, wherein a first portion of a separator is configured to couple to an angled channel of the first tissue retainer and a second portion of the separator is configured to couple to an angled channel of the second tissue retainer; and wherein the separator is configured to move a distal end of the first tissue retainer away from a distal end of the second tissue retainer when the separator is moved down the angled channels of the first tissue first tissue retainer and the second tissue retainer.

2. The surgical retractor of claim 1, further comprising a nerve root retractor configured to couple to a separator.

3. The surgical retractor of claim 1, further comprising a nerve root retractor configured to couple to the first tissue retainer.

4. The surgical retractor of claim 1, further comprising an optical cable configured to couple an illumination source to the first tissue retainer.

5. The surgical retractor of claim 1, further comprising an optical cable configured to couple an illumination source to the surgical retractor.

6. The surgical retractor of claim 1, wherein the first tissue retainer comprises an extender configured to allow the length of the first tissue retainer to be increased during use.

7. The surgical retractor of claim 1, wherein the first tissue retainer is coupled to the second tissue retainer by at least one member.

8. A retractor system for a spinal surgery procedure; comprising:
a surgical retractor configured to enlarge a surgical opening in a patient; and
a nerve root retractor configured to removable couple to the surgical retractor to allow the retention of dura of a patient to one side of a spinal column.

9. The retractor system of claim 8, wherein the surgical retractor comprises a pair of tissue retainers and a pair of separators, wherein insertion of the separators between the tissue retainers enlarges the surgical opening.

10. The retractor system of claim 9, wherein the nerve root retractor is configured to couple to a separator of the surgical retractor.

11. The retractor system of claim 9, wherein the nerve root retractor is configured to couple to a tissue retainer of the surgical retractor.

12. The retractor system of claim 8, wherein the surgical retractor comprises a set of keyways, and wherein the nerve root retractor comprises a protrusion configured to complement the keyways to allow the nerve root retractor to be removably coupled to the surgical retractor.

13. The retractor system of claim 8, wherein the nerve root retractor comprises a hook configure to engage a portion of the surgical retractor, and wherein the hood is slidable relative to an end of the nerve root retractor.

14. The retractor system of claim 8, wherein a retainer portion of the nerve root retractor is offset from an upper portion of the nerve root retractor.

15. A method of retraction during surgery, comprising:
placing a pair of tissue retainers in an opening in a patient;
coupling at least one separator to the tissue retainers; and
moving the separator downwards to separate distal ends of the tissue retainers, retract tissue and form a larger opening.

16. The method of claim 15, wherein a first tissue retainer of the pair of tissue retainers is coupled to a second tissue retainer of the pair of tissue retainers.

17. The method of claim 15, further comprising coupling a first separator to a second separator before moving the separators downwards to separate distal ends of the tissue retainers.

18. The method of claim 15, further comprising removably coupling a nerve root retractor to the separator.

19. The method of claim 15, further comprising removably coupling a nerve root retractor to one of the tissue retainers.

20. The method of claim 15, wherein moving the separator downwards comprises forcing the separator along angled channels in the tissue retainers.
